(19) United States
(12) Patent Application Publication
(10) Pub. No.: US 2022/0387324 A1
(43) Pub. Date: Dec. 8, 2022

(54) USE OF STARCH NANOPARTICLES LOADED WITH ACTIVE MOLECULES FOR AQUACULTURE

(71) Applicants: UNIVERSITE DE LILLE, LILLE (FR); CENTRALE LILLE INSTITUT, VILLENEUVE D'ASCQ (FR); UNIVERSITÉ POLYTECHNIQUE HAUTS-DE-FRANCE, VALENCIENNES (FR); CENTRE NATIONAL DE LA RECHERCHE SCIENTIFIQUE, PARIS (FR); YNCREA HAUTS DE FRANCE, LILLE (FR); UNIVERSITÉ DU LITTORAL CÔTE D'OPALE, DUNKERQUE (FR)

(72) Inventors: RABAH BOUKHERROUB, VILLENEUVE D'ASCQ (FR); SAMI SOUISSI, SAINT-MARTIN-BOULOGNE (FR); STEFKA LE FUR, VILLIERS-SUR-MARNE (FR)

(21) Appl. No.: 17/770,640

(22) PCT Filed: Oct. 20, 2020
(86) PCT No.: PCT/EP2020/079504

§ 371 Date: Apr. 21, 2022

(30) Foreign Application Priority Data
Oct. 21, 2019 (FR) 1911761

(51) Int. Cl. A61K 9/16; A23K 50/80; A23K 20/163; A23K 20/174; A61K 31/375; A61K 31/714; A61K 31/205; A61K 33/04; A61K 31/65; A61K 31/198; A61K 9/00; A61P 15/08

(52) U.S. Cl. CPC A61K 9/1652; A23K 50/80; A23K 20/163; A23K 20/174; A61K 31/375; A61K 31/714; A61K 31/205; A61K 33/04; A61K 31/65; A61K 31/198; A61K 9/0056; A61P 15/08

(57) ABSTRACT

The present invention relates to the use of a starch nanoparticle loaded with at least one active molecule of interest to feed zooplankton, in particular a living prey chosen from rotifers, brine shrimp and copepods. In particular, the invention relates to the use of this loaded starch nanoparticle to increase the size and/or density and/or egg-laying rate of this zooplankton, and/or the hatching rate of the eggs thereof

### OBJECT OF THE INVENTION

The present invention relates to the use of starch nanoparticles loaded with active molecules for feeding zooplankton intended to be used in aquaculture.

### BACKGROUND OF THE INVENTION

Aquaculture corresponds to the culture of aquatic organisms, such as fish, crustacea, mollusks, and aquatic plants. It can be carried out in fresh water or salt water, in controlled conditions. Aquaculture represents an alternative solution to overfishing and the disappearance of certain sought-after aquatic organisms owing to the increasing need for seafood. In 2008, it supplied 76.4% of the global consumption of freshwater fish, 64.1% of mollusks, 46.4% of shellfish and only 2.6% of seawater fish. Aquaculture also represents a solution for restocking rivers or ponds from which species have disappeared, in particular because of overexploitation. In 2016, the global output from aquaculture (according to the FAO) reached 110 million tonnes and thus exceeds the global output from fishing.

However, there are many drawbacks associated with aquaculture. At the local level, for example, traditional aquaculture (where fish are grown in cages or in tanks on the shoreline) may in certain cases cause more damage to the environment than wild fishing. Local problems are often related to waste treatment and the secondary effects of the antibiotics used. Aquaculture may also contribute to the proliferation of invasive species likely to damage fauna, as is the case for Nile perch or for species that escape from farms. The waste resulting from aquaculture and produced by fish are organic and consist of nutrients that are necessary for all the components of the aquatic food chain, but excessive supply of them at the bottom of the ocean may damage or even eliminate benthic life. They may also decrease the levels of oxygen dissolved in the water and affect wild fauna.

In addition, aquaculture requires large amounts of nutrients for breeding the aquatic species. For example, salmon farming requires a large amount of wild fish used as feed (i.e. food for farmed fish). In principle, fish cannot produce omega-3 or the fatty acids that their body needs. They accumulate them either by consuming microalgae (in the case of herring and sardine), or by consuming prey fish (in the case of salmon). Carnivorous fish such as salmon need large nutrient supplies of proteins derived from forage fish. The problem is that it takes several kilograms of forage fish to produce 1 kg of farmed salmon. While the demand for salmon continues to rise, the need for forage fish gets even larger. Thus, 75% of global fishing has exceeded or is about to exceed its maximum sustainable yield.

Aquaculture carried out in a manner that is not sustainable represents an increasing threat to coastal ecosystems. In fact, nearly 20% of the mangrove forests have been destroyed since 1980, partly due to intensive farming of prawns. More than 269 000 hectares of mangroves in the Philippines have been converted into prawn farms. Most of these farms are abandoned within a decade owing to the accumulation of toxins and loss of nutrients. Moreover, cost analysis has demonstrated that these farms were not economically viable.

Salmon farms pollute the ecosystems of the shorelines where they are installed. In fact, a farm with 200 000 tonnes of salmon produces as much fecal waste as a town with 60 000 inhabitants. This waste is discharged directly into the sea without being treated. There is also accumulation of heavy metals (in particular copper and zinc) on the benthos (sea bed) near the salmon farms. Wild fish are on the decline globally, as fish habitats, in particular in estuaries, are in a critical state. The farming of fish, such as salmon, does not solve the problem, as they need to consume products derived from other fish, such as fish meal and fish oil.

Several prospective analyses of the development of the aquaculture sector have emphasized the importance of innovation in all steps of the aquaculture production cycle. Thus, the current trend favors the creation of aquaculture farms inland based on water treatment technologies. We often talk of RAS systems (Recirculating Aquaculture Systems), which offer innovative solutions in the control of the quality of the water used and recycling thereof. The second major obstacle in aquaculture relates to the feeding of fish of all sizes. In fact, the current trend in research and R&D programs aims to test new plant-based foodstuffs for maximum limitation of fish meal and fish oil in the food matrix. However, the fishes domesticated in the past (salmon, sea bass, sea bream and tropical grouper) are carnivorous. Consequently, the choice of species requires considerable thought. In all cases, the first phases of the cycle of farming of a species (conventional or novel) after hatching of the eggs represent a major obstacle and a real challenge for technological development and innovation.

In fact, although aquaculture is the food growing system with one of the strongest growths in the world (8.3% per year, SOFIA report of the FAO 2010), its development is still limited owing to the difficulties in controlling the start of the life cycle of many fish species. The absence of suitable food is a major stumbling block in the production of larvae of many fish species, and very few fish species can be raised on an artificial diet without live food. Today, whereas the production of fish larvae of high quality is essential for aquaculture, for the aquarium trade and for purposes of storage, feeding of the larvae is increasing in importance in the area of aquaculture. Moreover, owing to a global decrease in the supply of brine shrimp (classical live prey harvested in the form of cysts in the natural habitat) and diversification of the species reared, such as ornamental fish with very small larvae, or other species with very small mouth openings, an alternative or a supplement to live food for the larvae is essential.

Although the juvenile and adult phase is very well under control in aquaculture using artificial feed (granules), rearing larvae is still problematic even for a high-performance hatchery working with just one species. In fact, a high mortality during the fish life cycle is observed during larval development. Traditionally, brine shrimp and rotifers serve as live food (live prey) for farmed fish in the larval stage. However, these species do not cover all the nutritional needs of fish; they are therefore often enriched with artificial industrial products, which are sometimes expensive. Moreover, the natural stocks of brine shrimp must not suffer from the current ever increasing demand from aquaculture.

That is why current research in aquaculture is concentrating on a sustainable, environmentally friendly solution for the production of commonly used live prey such as brine shrimp and rotifers. However, these classical live prey species, brine shrimp and rotifers, are poor from a nutritional standpoint and often require enrichment based on nutrients that are encapsulated before being given to the fish larvae to eat. These enrichment products are in particular emulsions and/or powders with low water solubility. Thus, significant losses of the enrichment products of live prey are often observed, which in addition quickly affect the water quality. Thus, extension to other species of live prey is an additional area of research to be developed. This applies, for example, to copepods, which represent an ideal solution as an alternative food for fish larvae. Copepods meet their nutritional needs and constitute a large part of their food in their natural environment.

Faced with these problems, some scientists have in particular been interested in nanoparticles as vectors or for administering active molecules of interest in the case of live prey. For example, Jimenez-Fernandes et al. (Aquaculture 432 (2014) 426-433) describe chitosan nanoparticles loaded with vitamin C administered directly to larvae of Senegalese sole (Solea senegalensis), but also to rotifers (Brachionus plicatilis).

However, there is still a real need to develop new means for the large-scale production of zooplankton in sufficient quantity and quality, intended to be used in aquaculture, while minimizing the environmental impact.

### SUMMARY OF THE INVENTION

In this context, the inventors have demonstrated, surprisingly, that starch nanoparticles loaded or functionalized with at least one active molecule of interest made it possible to increase the size and density of zooplankton more quickly, and more particularly in live prey such as rotifers, brine shrimp, and copepods, relative to unloaded starch nanoparticles and chitosan or alginate nanoparticles, whether loaded or not. The inventors have also demonstrated an effect of these loaded starch nanoparticles on the egg-laying rate and the hatching rate of the eggs of this live prey, in particular in copepods.

The present invention therefore relates to the use of a starch nanoparticle loaded with at least one active molecule of interest for feeding zooplankton. More particularly, the invention relates to the use of a starch nanoparticle loaded with at least one active molecule of interest for increasing the size and/or density and/or egg-laying rate of zooplankton, and/or the hatching rate of the eggs of zooplankton. Preferably, the zooplankton is a live prey selected from rotifers, brine shrimp, and copepods.

According to a particular embodiment, the starch nanoparticle loaded with at least one active molecule of interest has an average size between 10 and 500 nm, between 20 and 450 nm, between 40 and 400 nm, preferably of about 40, 80, 100, 200, or 400 nm, and even more preferably about 80 nm.

According to another particular embodiment, the proportion by weight of said at least one active molecule of interest is between 0.01% and 10%, between 0.01% and 5%, preferably between 0.1% and 4%, and even more preferably between 1 and 3% relative to the total weight of the starch nanoparticle.

According to another particular embodiment, said at least one active molecule of interest is selected from vitamins, carotenoids, antibiotics, hormones, minerals, amino acids, peptides, proteins, fatty acids, and derivatives thereof. Preferably, the active molecule of interest is selected from vitamin B12, vitamin C, betaine, selenium, tetracycline, trimethoprim, oxolinic acid, taurine, methionine, glutathione, iodine, estrogen and estradiol.

The invention further relates to a use of a composition comprising at least one starch nanoparticle loaded with at least one active molecule of interest as described in the present application and, optionally, a nutrient, in particular a microalga, for feeding zooplankton, preferably a live prey selected from rotifers, brine shrimp, and copepods. According to a particular embodiment, the composition is in the form of powder or solution. According to another particular embodiment, the composition comprises an amount of starch nanoparticles loaded with at least one active molecule of interest between 0.1 and 300 mg/L, between 1 and 200 mg/L, between 10 and 150 mg/L, between 10 and 100 mg/L, preferably between 10 and 80 mg/L, and even more preferably between 20 and 60 mg/L. According to another particular embodiment, the composition is applied daily.

The invention relates additionally to a composition comprising at least one starch nanoparticle loaded with betaine, at least one starch nanoparticle loaded with vitamin C, and at least one starch nanoparticle loaded with vitamin B12. It further relates to the use of said composition for feeding zooplankton, preferably a live prey selected from rotifers, brine shrimp, and copepods.

The invention also relates additionally to a method for enriching the nutritive value of zooplankton, preferably a live prey selected from rotifers, brine shrimp, and copepods, comprising the application of at least one starch nanoparticle loaded with at least one active molecule of interest or of a composition as defined in the present application.

### BRIEF DESCRIPTION OF THE FIGURES

FIG. 1:

FIG. 1A: Development of the size of brine shrimp as a function of the type of food.

FIG. 1B: Development of the size of brine shrimp as a function of the type of food.

FIG. 1C: Development of the size of brine shrimp as a function of the type of food.

FIG. 2:

FIG. 2A: Daily monitoring of the densities of rotifers as a function of the type of food.

FIG. 2B: Daily monitoring of the densities of rotifers as a function of the type of food.

FIG. 2C: Daily monitoring of the densities of rotifers as a function of the type of food.

FIG. 3: Daily monitoring of the number of eggs laid by female copepods as a function of the type of food.

FIG. 4: Hatching rate, at one month, of eggs laid by female copepods as a function of the type of food.

FIG. 5: Daily monitoring of the increase in length and width of the copepods as a function of the type of food.

FIG. 6: Survival of the eggs laid by female copepods at D13.

### DETAILED DESCRIPTION

As illustrated in the examples given hereunder, the inventors have demonstrated that starch nanoparticles loaded or functionalized with at least one active molecule of interest allowed a more rapid increase in the size, density, egg-laying rate, and hatching rate of zooplankton, and more particularly of live prey such as rotifers, brine shrimp, and copepods. These starch nanoparticles, loaded with at least one active molecule of interest, can thus act as vectors of nutrients for improving the nutritional profile and the performance of the zooplankton in aquaculture. Furthermore, these loaded starch nanoparticles are stable in salt water (seawater). They are used in relatively small amounts, thus limiting the environmental impact and the development of bacterial resistance. They are also nontoxic to zooplankton. They are applicable to various species of zooplankton such as brine shrimp, rotifers, and copepods, regardless of the size of their mouth, as the diameter of these nanoparticles can easily be controlled. They offer a multitude of treatment possibilities because they can be loaded or functionalized with one or more active molecules of interest. Finally, their cost of manufacture is relatively low, as they are obtained from starch, which is a biopolymer that is readily available commercially, and the method of manufacture by simple grinding is suitable for an industrial scale.

The present invention therefore relates to a use of a starch nanoparticle loaded with at least one active molecule of interest for feeding zooplankton, preferably a live prey selected from rotifers, brine shrimp, and copepods. The invention also relates to a process or a method for feeding zooplankton, preferably a live prey selected from rotifers, brine shrimp, and copepods, comprising the application of at least one starch nanoparticle loaded with at least one active molecule of interest to said live prey.

The invention also relates to a use of a starch nanoparticle loaded with at least one active molecule of interest for enriching the nutritive value of zooplankton, preferably a live prey selected from rotifers, brine shrimp, and copepods. The invention also relates to a process or a method for enriching the nutritive value of zooplankton, preferably a live prey selected from rotifers, brine shrimp, and copepods, comprising the application of at least one starch nanoparticle loaded with at least one active molecule of interest or of a composition comprising at least one loaded starch nanoparticle as described in the present application.

According to a particular embodiment, the invention relates to a use of a starch nanoparticle loaded with at least one active molecule of interest for increasing the size and/or density and/or egg-laying rate of zooplankton, preferably a live prey selected from rotifers, brine shrimp, and copepods, and/or the hatching rate of the eggs of zooplankton. The invention also relates to a process or a method for increasing the size and/or density and/or egg-laying rate of zooplankton, preferably a live prey selected from rotifers, brine shrimp, and copepods, comprising the application of at least one starch nanoparticle loaded with at least one active molecule of interest or of a composition comprising at least one loaded starch particle as described in the present application.

### Starch Nanoparticles (NPs)

Starch is a polysaccharide, made up of chains of D-glucose molecules, representing the main energy source of all living beings. Starch is present in cereal grains (maize, wheat), legumes (pea), roots, tubers and rhizomes (potato, sweet potato, cassava), and fruits (banana). Its empirical formula is [Cₓ(H₂O)_{y})]ₙ.

Starch is a mixture of two homopolymers that differ in their degree of branching and their degree of polymerization: amylose, lightly branched with short branches, consisting of 600 to 1000 units of D-glucose, leading to a molecular weight of 10⁴ to 10⁶ dalton, and amylopectin, with long branches, comprising from 10 000 to 100 000 molecules of D-glucose, leading to a molecular weight of 10⁶ to 10⁸ dalton.

The starch nanoparticles (NPs), loaded with at least one active molecule of interest, used in the invention, may be prepared by any technique known by a person skilled in the art, and in particular by the “planetary grinding” technique. This technique offers the advantage of preparing starch NPs loaded with an active molecule of interest in large quantities and is therefore very suitable for application on an industrial scale. Furthermore, this technique also makes it possible to prepare NPs of different sizes depending on the grinding cycles used. The size of the loaded starch NPs may thus be adapted to the target species of live prey. Examples of grinding cycles are illustrated in Table 1 of example 1 hereunder.

According to a particular embodiment, the starch NPs, loaded with at least one active molecule of interest, have an average size between 10 and 500 nm, between 20 and 450 nm, between 40 and 400 nm, preferably of about 40, 80, 100, 200, or 400 nm, and even more preferably about 80 nm.

According to the invention, the starch NPs are loaded with at least one active molecule of interest. “Loaded” means that the starch NPs comprise at least one active molecule of interest. It is also to be understood that the starch NPs are functionalized with at least one active molecule of interest. “At least one active molecule of interest” means that the starch NPs are loaded necessarily. They may therefore comprise a plurality of combinations of different active molecules of interest preferably numbering at least between 1 and 10, between 1 and 5, and even more preferably between 1 and 3. According to a preferred embodiment, the starch NPs are loaded with one, two, or three different active molecules of interest.

Obviously, the amount by weight of active molecule(s) of interest per particle may be adjusted as a function of the amount of active molecule(s) of interest that is to be delivered, given or administered to the live prey. Thus, if we wish to deliver a relatively large amount to the live prey, the starch NP will have a larger load of active molecule(s) of interest.

According to a particular embodiment, the starch NP is loaded with at least one active molecule of interest, in which the proportion by weight of the active molecule of interest is between 0.01% and 10%, between 0.01% and 5%, preferably between 0.1% and 4%, and even more preferably between 1 and 3% relative to the total weight of the starch nanoparticle. According to an even more preferred embodiment, the starch NP is loaded with at least one active molecule of interest at 1 wt %, relative to the total weight of the starch NP. According to another even more preferred embodiment, the starch NP is loaded with at least one active molecule of interest at 3 wt %, relative to the total weight of the starch NP.

“Active molecule of interest” means any biologically active molecule usable in aquaculture, and more particularly any molecule having an effect on zooplankton, in particular live prey such as brine shrimp, rotifers, and copepods. As “active molecule of interest”, we may mention, for example and without limitation, vitamins, carotenoids, antibiotics, hormones, minerals, amino acids, peptides, proteins, fatty acids, and derivatives thereof.

Vitamins are organic substances necessary for a living organism's metabolism. They may also be synthesized in sufficient quantity by the living organism itself. An insufficient intake or absence of a vitamin may be the cause of many diseases. Conversely, an excessive intake of vitamins may be toxic for the living organism. Vitamins are generally divided into two groups: water-soluble vitamins and fat-soluble vitamins. Among the water-soluble vitamins, we may mention vitamin B1 (thiamine), vitamin B2 (riboflavin), vitamin B3 (nicotinamide, niacin), vitamin B5 (pantothenic acid), vitamin B6 (pyridoxine), vitamin B8 (biotin), vitamin B9 (folic acid), vitamin B12 (or cobalamin), and vitamin C (ascorbic acid). Among the fat-soluble vitamins, we may mention vitamin A (retinol), vitamin D (calciferol), vitamin E (tocopherol), vitamin K1 (phylloquinone), and vitamin K2 (menaquinone).

Antibiotics are natural or synthetic substances that destroy or block the growth of bacteria. There are several classes of antibiotics including, in particular, aminoglycosides, beta-lactam antibiotics, cyclins, glycopeptides, macrolides, nitrofurans and nitroimidazoles, quinolones, phenicols, polypeptides, and sulfamides. Without limitation, we may mention as examples of antibiotics, penicillin, cephalosporins, tetracycline, oxytetracycline, gentamicin, trimethoprim, oxolinic acid, flumequine, rifampicin, chloramphenicol and florfenicol.

Hormones are biologically active chemical substances synthesized by glandular cells. Hormones transmit a message in chemical form and thus play a role of messenger in the organism. As examples of hormones and derivatives, we may mention, without limitation, estrogen and estradiol, and the thyroid hormones such as thyroxine.

Examples of minerals are, without limitation, iodine, selenium, phosphorus, calcium, magnesium, iron, copper, zinc, manganese, sodium, and potassium. In the context of the present invention, the term “minerals” also includes inorganic compounds, such as potassium iodide (KI), potassium iodate (KIO₃), sodium iodide, and sodium selenite.

Examples of amino acids or amino acid derivatives are, without limitation, methionine, taurine, betaine, aspartic acid, threonine, serine, glutamic acid, proline, cysteine, glycine, alanine, valine, lysine, histidine, leucine, tyrosine, phenylalanine, and isoleucine.

Examples of peptides or peptide derivatives are, without limitation, glutathione, epinicidin, cristine, and bacitracin.

Fatty acids are carboxylic acids with aliphatic chains. They are important sources of metabolic energy. Examples of fatty acids are, for example, butyric acid, valeric acid, caprylic acid, capric acid, lauric acid, myristic acid, palmitic acid, stearic acid, arachidic acid, behenic acid, lignoceric acid, myristoleic acid, palmitoleic acid, oleic acid, vaccenic acid, linoleic acid, alpha-linoleic acid, arachidonic acid, eicosapentaenoic acid, erucic acid, and docosahexaenoic acid.

According to a particular embodiment of the invention, said at least one active molecule of interest is selected from vitamins, carotenoids, antibiotics, hormones, minerals, amino acids, peptides, proteins, fatty acids, and derivatives thereof. According to a preferred embodiment of the invention, said at least one active molecule of interest is selected from vitamin B12, vitamin C, betaine, selenium, tetracycline, trimethoprim, oxolinic acid, taurine, methionine, glutathione, iodine, estrogen and estradiol.

### Composition

The invention relates to the use of a composition comprising at least one starch nanoparticle loaded with at least one active molecule of interest as defined in the present application and, optionally, a nutrient, in particular a microalga, for feeding zooplankton, preferably a live prey selected from rotifers, brine shrimp, and copepods.

The composition may thus comprise one or more starch NPs loaded with at least one active molecule of interest. When the composition comprises a number of at least two starch NPs loaded with at least one active molecule of interest, it may be said that the composition comprises a set of loaded starch NPs.

According to a particular embodiment of the invention, the composition comprises a set of starch nanoparticles loaded with an active molecule of interest. According to this embodiment, the set of starch NPs is loaded with the same active molecule of interest. Examples of compositions according to this particular embodiment are, for example, a composition comprising a set of starch NPs loaded with vitamin B12, a composition comprising a set of starch NPs loaded with betaine, a composition comprising a set of starch NPs loaded with vitamin C.

According to another particular embodiment of the invention, the composition comprises a set of starch nanoparticles, each loaded with at least two active molecules of interest. According to this embodiment, the set of starch NPs is loaded with at least two different active molecules. Examples of compositions according to this particular embodiment are, for example, a composition comprising a set of starch NPs loaded both with vitamin B12 and with betaine, a composition comprising a set of starch NPs loaded both with vitamin C and with betaine, and a composition comprising a set of starch NPs loaded both with vitamin B12, with vitamin C, and with betaine.

According to another particular embodiment, the composition comprises several sets of nanoparticles as described above. Preferably, the composition comprises between 2 and 10, 2 and 9, 2 and 8, 2 and 7, 2 and 6, 2 and 5, 2 and 4, and even more preferably 2, 3, or 4 sets of loaded starch nanoparticles. Examples of compositions according to this particular embodiment are, for example, a composition comprising a set of starch NPs loaded with vitamin B12 and a set of starch NPs loaded with betaine; a composition comprising a set of starch NPs loaded with vitamin C and a set of starch NPs loaded with betaine; a composition comprising a set of starch NPs loaded with vitamin B12, a set of starch NPs loaded with vitamin C, and a set of starch NPs loaded with betaine; a composition comprising a set of starch NPs loaded both with vitamin B12 and with vitamin C and a set of starch NPs loaded with betaine; a composition comprising a set of starch NPs loaded both with betaine and with vitamin C and a set of starch NPs loaded with vitamin B12; and a composition comprising a set of starch NPs loaded both with vitamin B12 and with betaine and a set of starch NPs loaded with vitamin C.

Of course, the active molecules of interest used in the above examples of compositions are interchangeable and may be replaced with any active molecule of interest as defined in the present application.

The invention further relates to a composition comprising a set of starch nanoparticles loaded with at least two active molecules of interest as described in the present application, including the particular, preferred embodiments.

The invention further relates to a composition comprising at least two sets of starch nanoparticles loaded with at least one active molecule of interest as described in the present application, including the particular, preferred embodiments.

A preferred composition according to the invention comprises at least one starch nanoparticle loaded with betaine, at least one starch nanoparticle loaded with vitamin C, and at least one starch nanoparticle loaded with vitamin B12. This preferred composition therefore comprises a set of starch nanoparticles loaded with betaine, a set of starch nanoparticles loaded with vitamin C, and a set of starch nanoparticles loaded with vitamin B12.

According to a particular embodiment, the composition as defined in the present application further comprises a nutrient, in particular a microalga. Examples of microalgae are T-isochrisis (T-iso) and Rhodomonas (Rhodo).

According to another particular embodiment, the composition is in the form of powder or solution. Preferably, the composition is in the form of solution.

According to another particular embodiment, the composition comprises an amount of starch nanoparticles loaded with at least one active molecule of interest between 0.1 and 300 mg/L, between 1 and 200 mg/L, between 10 and 150 mg/L, between 10 and 100 mg/L, preferably between 10 and 80 mg/L, and even more preferably between 20 and 60 mg/L. In the context of the invention, the amount of starch nanoparticles loaded with at least one active molecule of interest is expressed in weight/volume. More specifically, it is expressed in mg/L of water. Preferably, the water is salt water, such as seawater.

An even more preferred composition according to the invention comprises about 30 mg/L of starch NPs loaded with betaine at 3%, about 5 mg/L of starch NPs loaded with vitamin B12 at 3%, and about 5 mg/L of starch NPs loaded with vitamin C at 3%.

Another even more preferred composition according to the invention comprises about 200 mg/L of starch NPs loaded with betaine at 3%, about 200 mg/L of starch NPs loaded with vitamin B12 at 3%, about 200 mg/L of starch NPs loaded with vitamin C at 3%, and about 200 mg/L of starch NPs loaded with selenium at 3%.

Another even more preferred composition according to the invention comprises about 30 mg/L of starch NPs loaded with betaine at 1% or 3%, about 5 mg/L of starch NPs loaded with vitamin B12 at 1% or 3%, and about 5 mg/L of starch NPs loaded with vitamin C at 1% or 3%.

A preferred object therefore relates to such a composition for feeding zooplankton, preferably a live prey selected from rotifers, brine shrimp, and copepods.

### Live Prey

According to the invention, the starch NPs loaded with at least one active molecule of interest and the compositions as described in the present application are used for feeding zooplankton, and in particular, live prey used in aquaculture, such as rotifers, brine shrimp, and copepods.

The live prey used in aquaculture are also called “zooplankton”. Zooplankton covers the animal part of plankton and represents the organisms living in the column of water in any aquatic environments (fresh water, brackish water and seawater) with limited capacity for locomotion. Zooplankton is classified according to the size of the organisms. We may mention in particular the class of microzooplankton (<200 μm) comprising rotifers, the first larval stages (nauplii) of copepods, protists, ciliates and planktonic foraminifers. We may also mention the class of mesozooplankton comprising the juvenile and adult stages of copepods, free crustaceans, copepods that live in any aquatic environments and cladocerans (e.g. Daphnia) occurring essentially in freshwater environments. Zooplankton also comprises other classes of organisms of large size (macro- and megazooplankton) such as certain crustaceans (krill and Mysidacea) and gelatinous zooplankton comprising mollusks, the Cnidaria (e.g.

jellyfish), and ctenophores. Finally, the majority of the larval stages of aquatic animals develop in planktonic form and form part of the meroplankton, as opposed to the holoplankton, which includes all organisms whose entire life cycle proceeds in the column of water.

Brine shrimp (Artemia salina) are crustaceans living in salt lakes or salt marshes (often in Latin America and California). A specific feature of brine shrimp is that they swim on their back. They are able to live in water with very high salinity (up to about 300 g/L). Their adult size is on average from 8 to 10 mm but it may reach 15 mm, depending on their environment. Their body, of elongated shape, is divided into 20 segments and comprises 20 pairs of “legs”, or leaf-like appendages that oscillate with a regular rhythm. When the environment of the brine shrimp is unfavorable to their development, the female may produce dormancy eggs (diapause) called cysts. These cysts can be preserved for a long time without access to water. They can then be rehydrated to produce brine shrimp nauplii. The nauplii reach adult age after 2 weeks at a temperature of 25° C. Brine shrimp are the live prey most used in the breeding of fish for aquaria and in aquaculture.

A particular aspect of the invention relates to a use of a starch nanoparticle loaded with at least one active molecule of interest or a composition comprising at least one such starch nanoparticle for feeding brine shrimp. A preferred aspect of the invention relates to a use of a starch nanoparticle loaded with at least one active molecule of interest or a composition comprising at least one such starch nanoparticle to increase the size and/or density and/or egg-laying rate of brine shrimp, and/or the hatching rate of eggs of said brine shrimp. An even more preferred aspect of the invention relates to a use of a starch nanoparticle loaded with at least one active molecule of interest or a composition comprising at least one such starch nanoparticle to increase the size of brine shrimp.

Rotifers are metazoan planktons that represent a good proportion of the microzooplankton (zooplankton of small size). They inhabit various biotopes ranging from freshwater environments (fresh water) to hyper-salted environments (salt lakes, etc.). However, depending on the environments, they are not represented equally: they are very common in fresh and briny water, but they are less abundant in the sea. The species Brachionus plicatilis is a euryhaline rotifer that belongs to the Monogononitida order whose reproduction is essentially parthenogenetic. Stages of sexual reproduction may be triggered by exogenous stimuli (temperature, salinity, etc.) and/or endogenous stimuli (aging, etc.). The male of the Brachionus, and of the Monogononitida in general, is dwarf and has a large testis which occupies most of the body volume. In the natural habitat, these rotifers spend adverse periods in the form of resting eggs (diapause stage) resulting from sexual reproduction: this is a quiescent state. Once the conditions become compatible with the life of these animals, the eggs will hatch and the female rotifers will reproduce parthenogenetically to colonize the environment. In farming, the physicochemical parameters are controlled and regulated in order to promote parthenogenesis. This is a mode of reproduction used by certain metazoan organisms. The females will produce eggs with 2n chromosomes, which will develop without fertilization by a male gamete. Parthenogenesis is equivalent to cloning. The individuals that come from one and the same female are all females, identical to each other and to their mother. In aquaculture (especially in hatcheries) and in aquariology (the sections devoted to live prey in aquaria), the rotifers are commonly used for feeding fish larvae during the first stages of development.

A particular aspect of the invention relates to a use of a starch nanoparticle loaded with at least one active molecule of interest or a composition comprising at least one such starch nanoparticle for feeding rotifers. A preferred aspect of the invention relates to a use of a starch nanoparticle loaded with at least one active molecule of interest or a composition comprising at least one such starch nanoparticle to increase the size and/or density and/or egg-laying rate of rotifers, and/or the hatching rate of the eggs of said rotifers. An even more preferred aspect of the invention relates to a use of a starch nanoparticle loaded with at least one active molecule of interest or a composition comprising at least one such starch nanoparticle to increase the density of rotifers.

The term copepod derives from two Greek roots: kope, meaning oar, and podos, meaning foot. There are more than 20 000 species of copepods, the majority of which are marine. These small crustaceans represent one of the main components of zooplankton (60%) and they play a key role in the food webs of the aquatic environment. They are primary and secondary consumers and also serve as food for many invertebrates and fish larvae. Their lifestyle is very variable depending on the species; they may be planktonic, epibenthic and benthic or else may live attached to a host, like a parasite. The diet also depends on the species: herbivorous, omnivorous and carnivorous. Reproduction is by mating as the gametes are not expelled into the environment. Reproduction is diurnal but it is influenced by external factors such as temperature, amount of food, salinity or the photoperiod. During mating, the male deposits a spermatophore on the urosome of the female. This is retained by the female to fertilize the eggs and incubate them in an egg sac or release them directly into the environment. Fertility is high and approximately corresponds to between 20 and 40 eggs per day for example in the species Acartia tonsa. When the environmental conditions become unfavorable, diapause eggs may be observed. These will wait for a more favorable period for hatching. The life cycle is complex as it consists of many molts and metamorphoses before reaching the reproductive adult stage. However, despite their complexity, these organisms have a considerable capacity for adaptation.

A particular aspect of the invention relates to a use of a starch nanoparticle loaded with at least one active molecule of interest or a composition comprising at least one such starch nanoparticle for feeding copepods. A preferred aspect of the invention relates to a use of a starch nanoparticle loaded with at least one active molecule of interest or a composition comprising at least one such starch nanoparticle for increasing the size and/or the egg-laying rate of copepods, and/or the hatching rate of the eggs of said copepods.

Preferably, the starch NPs loaded with at least one active molecule of interest or the compositions comprising said NPs as described in the present application are applied daily (once per day) on zooplankton, preferably a live prey selected from rotifers, brine shrimp, and copepods.

The invention therefore also relates to a daily use of at least one starch nanoparticle loaded with at least one active molecule of interest or of a composition comprising them, for feeding zooplankton, preferably a live prey selected from rotifers, brine shrimp, and copepods.

The invention further relates to a use of a composition comprising a dose of starch nanoparticles as described in the present application between 0.1 and 300 mg/L/day, between 1 and 200 mg/L/day, between 10 and 150 mg/L/day, between 10 and 100 mg/L/day, preferably between 10 and 80 mg/L/day, and even more preferably between 20 and 60 mg/L/day.

The invention further relates to a process or a method for feeding zooplankton, preferably a live prey selected from rotifers, brine shrimp, and copepods, comprising daily application of at least one starch nanoparticle loaded with at least one active molecule of interest or of a composition comprising them to said live prey.

The invention further relates to a process or a method for feeding zooplankton, preferably a live prey selected from rotifers, brine shrimp, and copepods, comprising application of a composition comprising a dose of starch nanoparticles as described in the present application between 0.1 and 300 mg/L/day, between 1 and 200 mg/L/day, between 10 and 150 mg/L/day, between 10 and 100 mg/L/day, preferably between 10 and 80 mg/L/day, and even more preferably between 20 and 60 mg/L/day.

The invention will be better understood in light of the following examples, which are given purely for illustration and are not limiting.

### EXAMPLES

### Example 1: Methods of Preparation and Characterization

1. Starch Manoparticles (Starch NPs)

2 g of starch was weighed in a 50 mL grinding bowl. Depending on the desired size of nanoparticles (NPs), zirconium oxide beads with a diameter of 10 mm or 20 mm were added to the bowl. The bowl was then placed in a PM100 grinding mill with a cycle defined according to the size of the NPs (see Table 1). 20 minutes before the end of the last cycle, the molecule of interest was introduced into the grinding bowl (except for the 400 nm NPs, the molecule of interest is introduced directly with the starch), weighed beforehand in an Eppendorf using a balance, at a concentration of 1% (20 mg in this case, for 2 g of NPs).

Once the cycle had ended, the bowl was put under a hood and the NPs contained in the bowl were transferred to a closed tube. The tubes were then stored in a refrigerator at 4° C. in the dark, to prevent the molecules of interest degrading.

TABLE 1

| Size of the | Grinding | | Number of | Bead size |
|---|---|---|---|---|
| NPs (nm) | time | | beads | (mm) |
| 40 | 2 | h | 10 | 10 |
| | 30 | min | 4 | 10 |
| 80 | 2.5 | h | 2 | 20 |
| 100 | 1.5 | h | 2 | 20 |
| 200 | 40 | min | 3 | 20 |
| 400 | 10 | min | 4 | 10 |

During the grinding cycles, a pause of 3 minutes was applied every 10 minutes and the direction of rotation of grinding was reversed. The grinding speed was 400 rpm.

2. Compositions

A solution of NPs was prepared by weighing an exact amount of NPs in a tube, using a balance under a hood (the amount to be weighed varies as a function of the species or the concentrations to be administered). Seawater was then added to the tube. This inhomogeneous solution was treated in the vortex and for some minutes with ultrasound until it had become homogeneous. Finally, the solution was poured into a culture beaker comprising live prey, or stored at 4° C. for some days, the time of the experiments.

### Example 2: Experimental Results on Brine Shrimp

The purpose of the experiments was to confirm the positive effects of the functionalized starch nanoparticles in aquaculture and aquariology, by monitoring the increase in size of the nauplii of brine shrimp, a species much used in this sector.

This study was carried out in the following conditions: temperature: 25° C., salinity: 25 g/L, pH: 7.9-8.3, water filtered and sterilized by autoclaving, aeration by Pasteur pipettes, illumination: 8 h to 21 h, ammonia content<1 mg/L, dissolved oxygen>4 mg/L.

1. Protocol:

For manipulation of 12 beakers of 1 L, the protocol was as follows:

Day 0: Hatching: 0.5 g of brine shrimp cysts were placed in 1L of seawater, with vigorous bubbling at a temperature of 25° C. for 48 h.

Day 2: Separation: The brine shrimp nauplii were recovered with the aid of a light beam, and separated into several 1 L beakers depending on the type of food, with moderate bubbling. 4 treatments were carried out per step with 3 replicas per type of food, which makes a total of 12 beakers of 1 L comprising about 300 brine shrimp per beaker.

Day 4: Feeding: Starting from the 4th day, 50 mg of NPs in the form of a homogeneous solution and 10 mL of T-iso algae were added to the beakers of 1 L of seawater every day.

Day 5, 7, 9, 11: Measurement of size: 25 brine shrimp were taken from each beaker, and fixed in alcohol. Fixation was carried out as follows: with a lamp or a light beam, the brine shrimp were attracted to the surface of the beaker, which made it easier to capture them with a Pasteur pipette. Then they were put in a Petri dish and ethanol was added (at a level of 5 to 10% of the total volume). Finally, once the brine shrimp were completely fixed, their size was measured with an ocular camera and Top View software, every 2 days for 11 days.

For steps 1 to 11 described below, 50 mg of NPs per liter of water was used for feeding the brine shrimp every day. The starch NPs had an average size of 80 nm, those of alginate about 250 nm and those of chitosan about 250 nm.

2. Results:

Step 1:

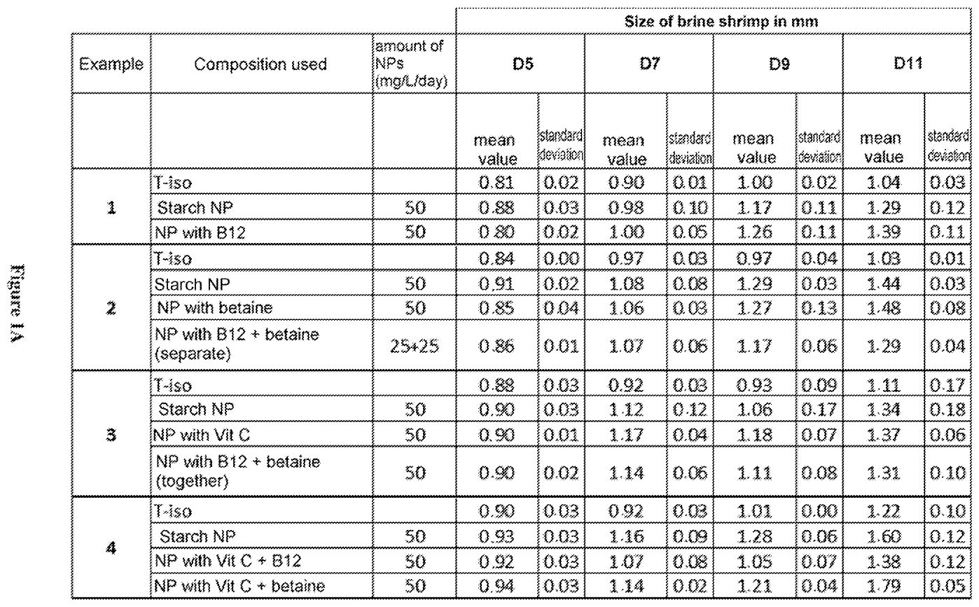

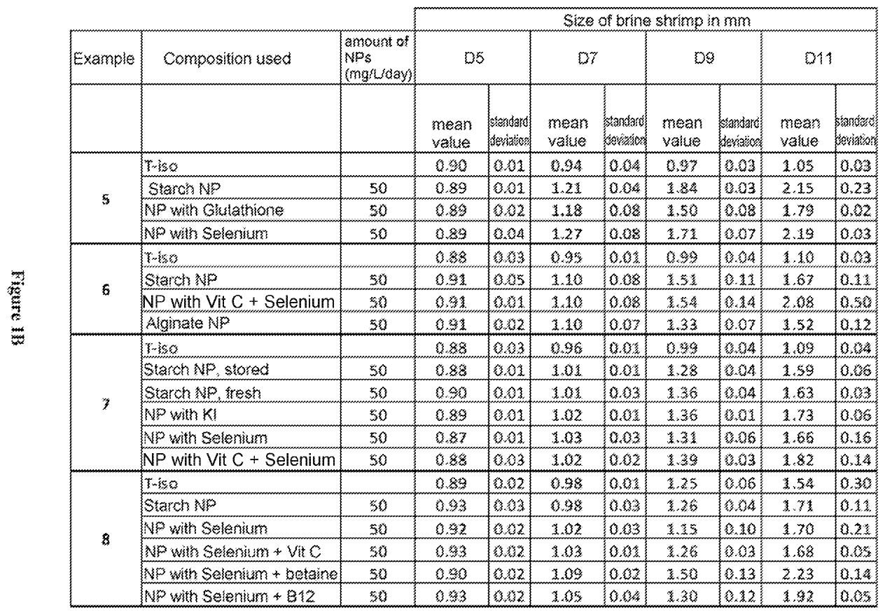

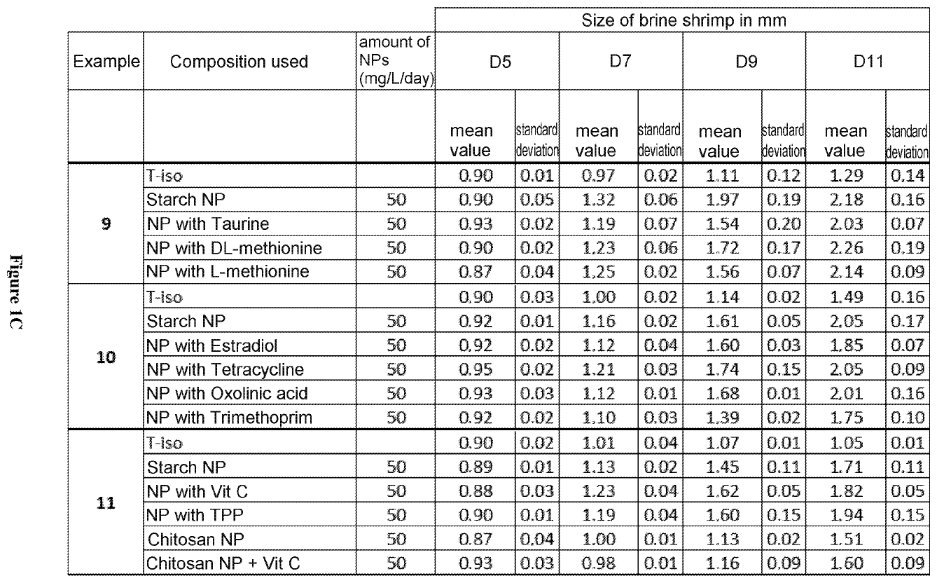

The results of the table in FIG. 1, Example 1, show that for the brine shrimp fed with starch NPs loaded with vitamin B12 at 0.1%, their size increases more quickly relative to brine shrimp fed with T-iso algae or with starch NPs not loaded with an active molecule of interest.

Step 2:

The results of the table in FIG. 1, Example 2, show that for the brine shrimp fed with starch NPs loaded with betaine at 1% and with unloaded starch NPs, their size increases more markedly relative to brine shrimp fed with T-iso algae or with a mixture of starch NPs loaded with vitamin B12 at 1% and starch NPs loaded with betaine at 1%.

Step 3:

The results of the table in FIG. 1, Example 3, show that for the brine shrimp fed with starch NPs loaded with vitamin C at 1%, with starch NPs loaded both with vitamin B12 at 1% and with betaine at 1%, and with unloaded starch NPs, their size increases more markedly relative to brine shrimp fed with T-iso algae.

Step 4:

The results of the table in FIG. 1, Example 4, show that for the brine shrimp fed with starch NPs loaded both with vitamin C at 1% and with betaine at 1%, their size increases more markedly relative to brine shrimp fed with T-iso algae, with the starch NPs alone, or with starch NPs loaded both with vitamin C at 1% and vitamin B12 at 1%.

Step 5:

The results of the table in FIG. 1, Example 5, show that for the brine shrimp fed with starch NPs loaded with selenium at 1% and with NPs with starch alone, their size increases more markedly relative to brine shrimp fed with T-iso algae or with the NPs loaded with glutathione at 1%.

Step 6:

The results of the table in FIG. 1, Example 6 show that for the brine shrimp fed with starch NPs loaded both with vitamin C at 1% and with selenium at 1%, their size increases more markedly relative to brine shrimp fed with T-iso algae or with the starch NPs. Furthermore, for the brine shrimp fed with NPs with starch alone, their size increases more markedly relative to brine shrimp fed with the alginate NPs.

Step 7:

The results of the table in FIG. 1, Example 7 show that for the brine shrimp fed with starch NPs loaded both with vitamin C at 1% and with selenium at 1% and with starch NPs loaded with potassium iodide at 1%, their size increases more markedly relative to brine shrimp fed with T-iso algae or with the starch NPs alone (fresh or stored). Furthermore, no difference is observed between the fresh starch NPs and the starch NPs stored for a month at 4° C. The starch NPs can therefore be stored without affecting the growth of brine shrimp.

Step 8:

The results of the table in FIG. 1, Example 8 show better results for the size of brine shrimp fed with starch NPs loaded both with selenium at 1% and with betaine at 1%.

Step 9:

The results of the table in FIG. 1, Example 9 show better results for the size of brine shrimp fed with starch NPs loaded with DL-methionine or with NPs with starch alone.

Step 10:

The results of the table in FIG. 1, Example 10 do not show better effects on the size of brine shrimp fed with starch NPs loaded with hormone or with antibiotic.

Step 11:

The results of the table in FIG. 1, Example 11 show better results for the size of brine shrimp fed with NPs with starch alone or loaded with an active molecule of interest relative to brine shrimp fed with NPs of chitosan alone or loaded with vitamin C at 1%. Furthermore, it was observed that the chitosan NPs increase the mortality of brine shrimp and make the seawater turbid in the beakers containing these chitosan NPs.

### Example 3: Experimental Results on Rotifers

The purpose of the experiments is to compare the development of the populations of rotifers belonging to the species Brachionus plicatilis, depending on the addition of different nanoparticles.

This study was carried out in the following conditions: temperature: 23-25° C., salinity: 25 g/L, pH: 7.9-8.3, water filtered and sterilized with sodium hypochlorite, aeration by Pasteur pipettes, illumination: 16 h/8 h, ammonia content<1 mg/L, dissolved oxygen>4 mg/L.

1. Preparation of the Parent Culture of Rotifers

The rotifers were cultured in 30-liter cylindrical tanks. The tanks were inoculated with low densities (10-30 individuals per ml) and fed every day with T-isochrisis and Rhodomonas sp to satiety. The water in the tanks was renewed completely once a week.

2. Tests on NPs Containing a Single Molecule of Interest

Protocol:

24 beakers of 1 L of culture of rotifers were set up. These 24 beakers were distributed into 6 groups (T-iso alga (control), starch alone, vitamin B12, betaine, vitamin C and selenium) consisting of 4 replicas. 100 mg of nanoparticles functionalized with 1% of a molecule of interest was introduced each day.

Results:

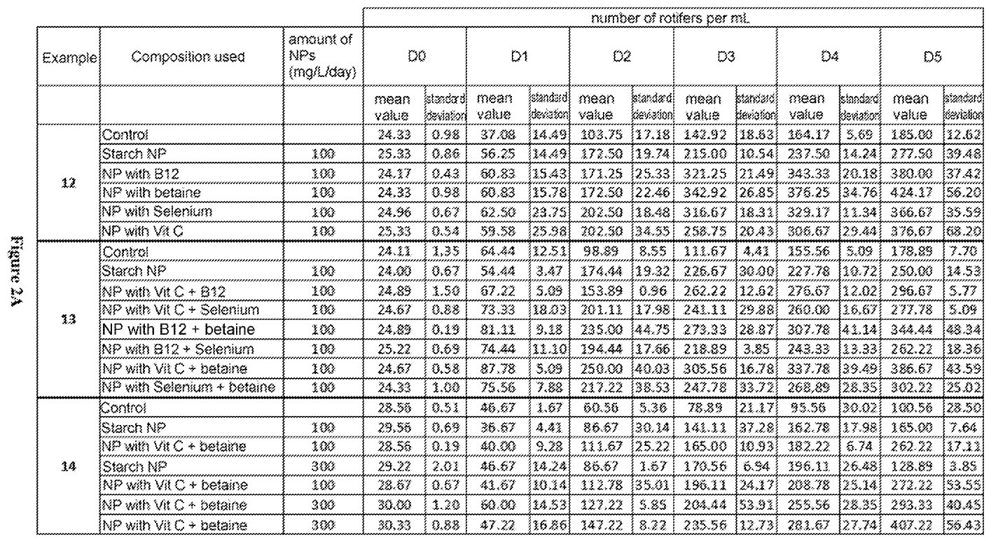

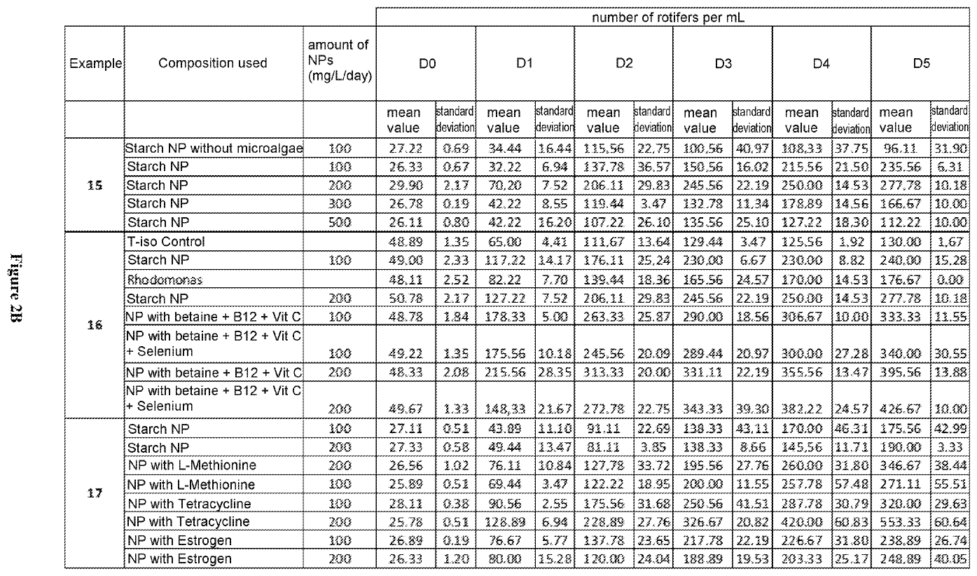

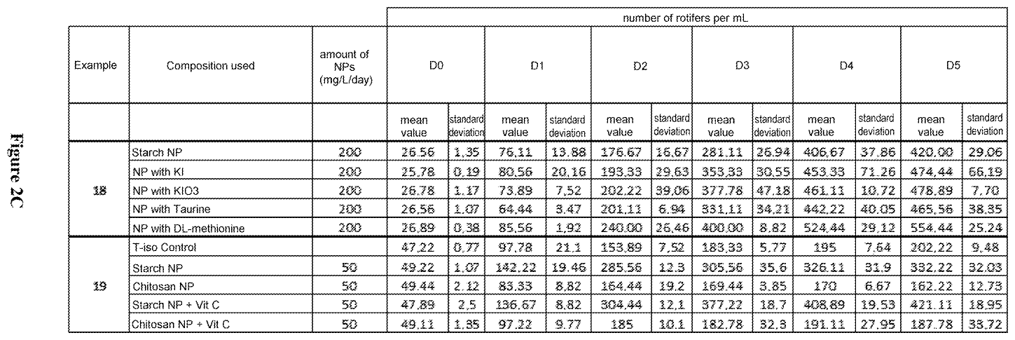

The results of the table in FIG. 2, Example 12 show that the best density is obtained with the rotifers fed with starch NPs loaded with betaine at 1%. Next come the starch NPs loaded with vitamin B12 at 1%, the starch NPs loaded with vitamin C at 1%, and the starch NPs loaded with selenium at 1%.

3. Tests on NPs Containing Two Molecules of Interest

Protocol:

24 beakers of 1L of culture of rotifers were set up. These 24 beakers were distributed into 8 groups (T-iso alga (control), starch alone, vitamin C and vitamin B12, selenium and vitamin C, vitamin B12 and betaine, selenium and vitamin B12, vitamin C and betaine, selenium and betaine) consisting of 3 replicas. 100 mg of nanoparticles functionalized with 1% of two molecules of interest was introduced each day.

Results:

The results presented in the table in FIG. 2, Example 13 show that the best density is obtained with the rotifers fed with starch NPs loaded both with betaine at 1% and with vitamin C at 1%. It is also observed that the best results are obtained with starch NPs loaded at least with betaine at 1%.

4. Tests on NPs Containing Two Molecules of Interest

Protocol:

The protocol used is identical to the protocol described above.

Results:

The results of the table in FIG. 2, Example 14 show that the best density is obtained with the rotifers fed daily with starch NPs at a concentration of 300 mg/L loaded with betaine and vitamin C at 3%.

5. Tests on NPs Without a Molecule of Interest

Protocol:

The protocol used is identical to the protocol described above without the molecules of interest.

Results:

The results of the table in FIG. 2, Example 15, show that the best density is obtained with the rotifers fed with starch NPs at a concentration of 200 mg/L per day. A higher dosage (300 and 500 mg/L/day) has a negative effect on the development of the rotifers. Note also that the treatment without microalgae gives negative results. The starch NPs alone therefore do not meet the nutritional needs of the rotifers.

6. Tests on NPs for the best combination

Protocol:

The protocol used is identical to the protocol described above.

Results:

The results of the table in FIG. 2, Example 16 show that the best density is obtained with the rotifers fed with 200 mg/L/day of starch NPs loaded with betaine at 3%; with vitamin B12 at 3%, with vitamin C at 3%, and with selenium at 3%.

7. Tests on NPs with other molecules of interest in connection with aquaculture

Protocol:

The protocol used is identical to the protocol described above.

Results:

The results of the table in FIG. 2, Example 17 show that the best density is obtained with the rotifers fed with the starch NPs loaded with tetracycline.

The results of the table in FIG. 2, Example 18 show that the best density is obtained with the rotifers fed with the starch NPs loaded with DL-methionine 1%.

8. Comparative Tests

Protocol:

The protocol used is identical to the protocol described above.

Results:

The results of the table in FIG. 2, Example 19 do not show any effect for the chitosan NPs, which moreover alter the quality of the water. The starch NPs therefore have better performance and are applicable in the culture of rotifers.

### Example 4: Experimental Results on Copepods

The purpose of the experiments was to compare egg production of copepods belonging to the species Acartia tonsa, depending on the addition of different nanoparticles.

The eggs were collected daily and counted to compare the egg-laying rates with and without

NPs. For this, the eggs were put in an 1800-mL beaker and homogenized with vigorous bubbling. Then 5 mL was taken and counted, 3 times. The eggs were then stored at 3° C. in an anoxic environment in the dark. These eggs were then reused for determining comparative hatching rates. For determining these hatching rates, a known number of eggs was incubated in 3 replicas in 80-mL beakers fed with Rhodomonas and T-isochrisis. After incubation for 48 h at 18° C., the eggs that had not hatched were counted to find the hatching rate.

This study was carried out in the following conditions: salinity: 33 g/L, pH: 7.8-8.3, filtered water, illumination: 16 h/8 h, ammonia content<1 mg/L, dissolved oxygen>4 mg/L.

1. Effect of the NPs on the Egg-Laying Rate of Copepods

Step 1:

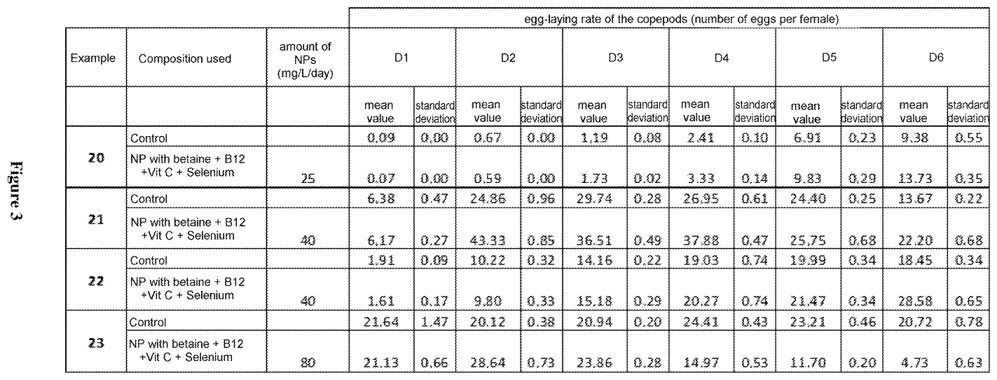

In this experiment a dose of 25 mg/L of NPs was added daily. This dose consists of 8.3 mg of starch NPs functionalized with betaine at 3%, 8.3 mg of starch NPs functionalized with vitamin C at 3% and 8.3 mg of starch NPs functionalized with vitamin B12, also at 3%. The results of the table in FIG. 3, Example 20 show an increase in the daily number of eggs laid per female by 20% on average with the functionalized NPs.

Step 2:

In this experiment, a composition of 40 mg/L of NPs comprising 30 mg of betaine at 3%, 5 mg of vitamin B12 at 3%, and 5 mg of vitamin C at 3% was added daily.

The results of the table in FIG. 3, Example 21 show an increase in the daily number of eggs laid per female by 44% on average with the functionalized NPs.

Step 3:

In this experiment, a composition of 40 mg/L of NPs comprising 30 mg of betaine at 1%, 5 mg of vitamin B12 at 1%, and 5 mg of vitamin C at 1% was added daily from D0 to D5. Then a composition of 40 mg of NPs comprising 30 mg of betaine at 3%, 5 mg of vitamin B12 at 3%, and 5 mg of vitamin C at 3% was added daily on D6.

The results of the table in FIG. 3, Example 22, show an increase in the daily number of eggs laid per female by 10% on average with the NPs functionalized at 1% and of 54% with the NPs functionalized at 3%.

Step 4:

In this experiment, a composition of 80 mg/L of NPs comprising 60 mg/L of betaine at 3%, 10 mg/L of vitamin B12 at 3%, and 10 mg/L of vitamin C at 3% was added daily.

The results of the table in FIG. 3, Example 23, show that the high dose (80 mg/L/d) of NPs affects the egg-laying rates starting from D4.

2. Effect of the NPs on the Hatching Rate of Copepods

After one month of storage, eggs of Acartia from three different collections were incubated in three replicas. A composition of 40 mg/L/d of NPs comprising 30 mg of betaine at 3%, 5 mg of vitamin B12 at 3%, and 5 mg of vitamin C at 3% had been added beforehand during culture of the parents.

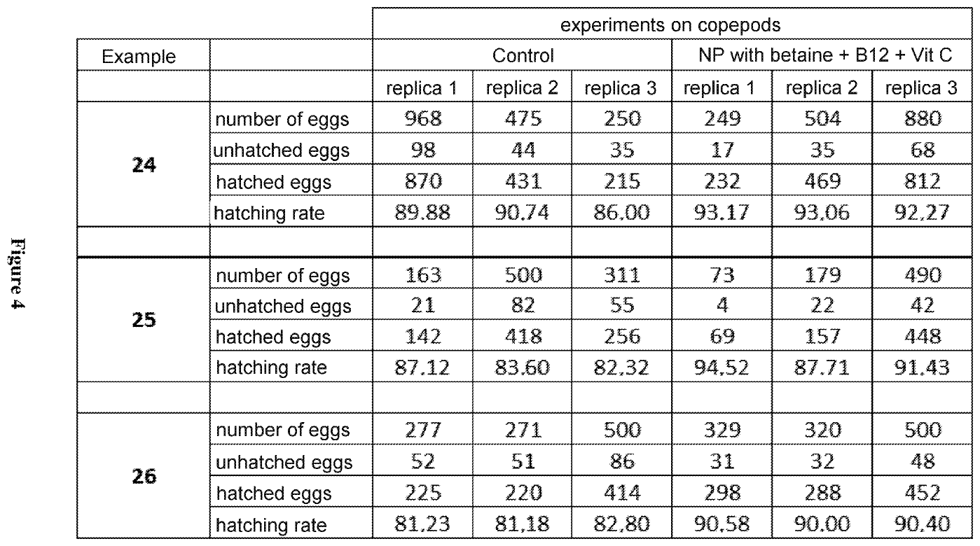

The results of the table in FIG. 4, Examples 24, 25 and 26 show that the functionalized NPs increase the hatching rate of copepod eggs by about 6%. Furthermore, better quality of the eggs was observed.

3. Effect on Survival and Growth of Copepods

Two 40 L columns were monitored. An identical number of eggs was left to incubate in these enclosures. One column received an addition of 10 mg/L of NPs functionalized at 3% of molecules of interest per day (8 mg of NPs with betaine, 1 mg of NPs with vitamin C and 1 mg of NPs with vitamin B12). Then 4 samples were taken, on the 3rd, 7th, 10th and 13th day for measuring the width and length of the copepods. 20 individuals were measured per treatment.

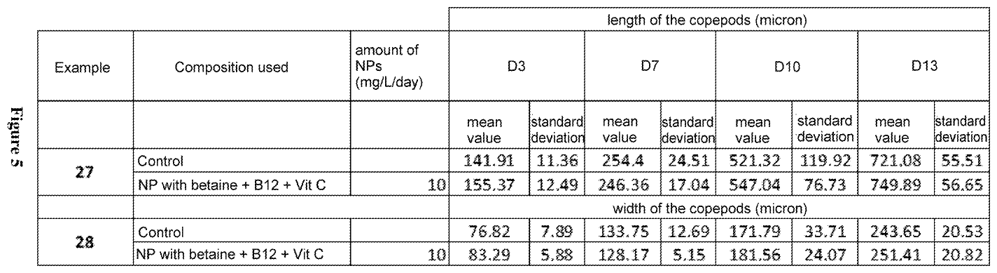

The results of the table in FIG. 5, examples 27 and 28 show a slight increase in width and length of the copepods with the functionalized NPs starting from D10.

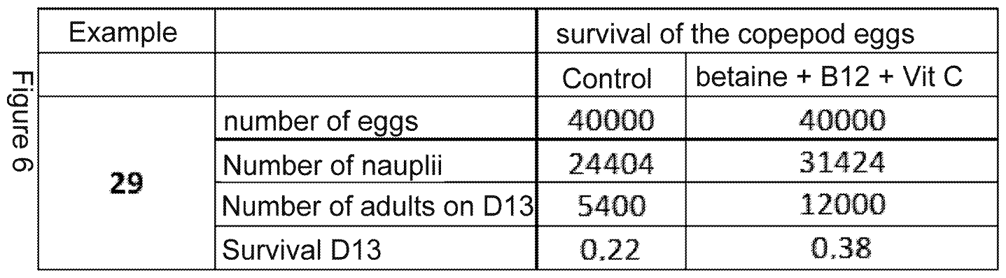

After a final count of the population, presented in the table in FIG. 6, Example 29, it was observed that survival at D13 is 22% (number of adults on D13/number of nauplii) for the control copepods versus 38% for the copepods treated with the functionalized NPs.

1-14. (canceled)

15. A method of feeding zooplankton comprising the application of at least one starch nanoparticle loaded with at least one active molecule of interest to said zooplankton.

16. The method according to claim 15, said method increasing the size and/or density and/or egg-laying rate of zooplankton, and/or the hatching rate of the eggs of zooplankton.

17. The method according to claim 15, in which the zooplankton is a live prey selected from the group consisting of rotifers, brine shrimp, and copepods.

18. The method according to claim 15, in which the starch nanoparticle loaded with at least one active molecule of interest has an average size between 10 and 500 nm.

19. The method according to claim 15, in which the proportion by weight of said at least one active molecule of interest is between 0.01% and 10% relative to the total weight of the starch nanoparticle.

20. The method according to claim 15, in which said at least one active molecule of interest is selected from the group consisting of vitamins, carotenoids, antibiotics, hormones, minerals, amino acids, peptides, proteins, fatty acids, and derivatives thereof

21. The method according to claim 15, in which said at least one active molecule of interest is selected from the group consisting of vitamin B12, vitamin C, betaine, selenium, tetracycline, trimethoprim, oxolinic acid, taurine, methionine, glutathione, iodine, estrogen and estradiol.

22. A method of feeding zooplankton comprising the application of a composition comprising at least one starch nanoparticle loaded with at least one active molecule of interest and, optionally, a nutrient to said zooplankton.

23. The method according to claim 22, in which the composition is in the form of powder or solution.

24. The method according to claim 22, in which the composition comprises an amount of starch nanoparticles loaded with at least one active molecule of interest between 0.1 and 300 mg/L.

25. The method according to claim 22, in which the composition is applied daily.

26. A composition comprising at least one starch nanoparticle loaded with betaine, at least one starch nanoparticle loaded with vitamin C, and at least one starch nanoparticle loaded with vitamin B12.

27. A method of feeding zooplankton comprising the application of a composition according to claim 26 to said zooplankton.

28. A method for enriching the nutritive value of zooplankton comprising the application of at least one starch nanoparticle loaded with at least one active molecule of interest and having an average size between 10 and 500 nm.

29. A method for enriching the nutritive value of zooplankton comprising the application of the composition according to claim 26 to said zooplankton.
